(19) United States
(12) Patent Application Publication
(10) Pub. No.: US 2020/0323793 A1
(43) Pub. Date: Oct. 15, 2020

(54) COMBINATION THERAPY COMPRISING A POLYUNSATURATED KETONE AND A CALCINEURIN INHIBITOR

(71) Applicant: Avexxin AS, Trondheim (NO)

(72) Inventors: Berit Johansen, Trondheim (NO); Astrid Jullumstrø Feuerherm, Trondheim (NO)

(73) Assignees: Avexxin AS, Trondheim (NO); Avexxin AS, Trondheim (NO)

(21) Appl. No.: 16/306,161

(22) PCT Filed: Jun. 5, 2017
(86) PCT No.: PCT/EP2017/063627

§ 371 Date: Nov. 30, 2018

(30) Foreign Application Priority Data
Jun. 3, 2016 (GB) 1609722.2
Mar. 17, 2017 (GB) 1704286.2

(51) Int. Cl. A61K 31/095; A61K 31/436; A61K 38/13

(52) U.S. Cl. CPC A61K 31/095; A61K 9/0014; A61K 38/13; A61K 31/436

(57) ABSTRACT

A synergistic pharmaceutical composition for simultaneous, parallel, sequential or separate use comprising a polyunsaturated ketone and a calcineurin inhibitor. The composition has utility in the treatment and prevention of skin disorders.

This invention relates to a pharmaceutical composition comprising certain polyunsaturated long-chain ketones in combination with certain calcineurin inhibitors such as pimecrolimus or a pharmaceutically acceptable salt, or a hydrate or solvate thereof. The invention also relates to the use of said pharmaceutical composition for the treatment or prevention of skin conditions such as dermatitis and psoriasis.

### BACKGROUND

This invention is concerned with a combination therapy for the treatment of certain skin conditions such as psoriasis and dermatitis. In its broadest sense, dermatitis is inflammation of the skin. It is a common and disfiguring skin condition which requires quick and efficient treatment. Dermatitis symptoms vary, however, with the different forms of the condition. Symptoms vary from skin rashes to bumpy rashes through to flaky skin and blisters. Although different types of dermatitis have varying symptoms, there are certain signs that are common for all of them, including redness of the skin, swelling, itching, skin lesions and sometimes oozing and scarring.

Also, the area of the skin on which the symptoms appear tends to be different with every type of dermatitis. Types of dermatitis are classified according to the cause of the condition. Contact dermatitis is caused by an allergen or an irritating substance. Irritant contact dermatitis accounts for 80% of all cases of contact dermatitis.

Atopic dermatitis is very common worldwide and increasing in prevalence. Atopic dermatitis is a type of eczema and is an inflammatory, chronically relapsing, non-contagious and itchy skin disorder.

Other less common forms of dermatitis include dermatitis herpetiformis. It is characterized by intensely itchy, chronic papulovesicular eruptions, usually distributed symmetrically on extensor surfaces such as the back of neck, scalp, elbows, knees, back, hairline, groin or face.

Seborrheic dermatitis is a dermatitis that occurs in the vicinity of sebaceous glands and is caused by sebum over production. The condition tends to give a scaly, flaky skin condition.

Stasis dermatitis is an inflammation on the lower legs which is caused by build-up of blood and fluid and it is more likely to occur in people with varicose veins.

Other common skin disorders include psoriasis. This is an autoimmune induced, chronic disease of skin characterised by red, itchy and scaly skin patches. Skin disorders in general and dermatitis and psoriasis in particular are disfiguring and can lead to reluctance of a sufferer to let people see their condition. Successful treatments of these skin disorders are therefore sought.

A common treatment for skin disorders is administration of one or more topical calcineurin inhibitors. The present inventors have now found that the combination of certain polyunsaturated ketones and certain calcineurin inhibitors such as pimecrolimus or a pharmaceutically acceptable salt, or a hydrate or solvate thereof results in a synergistic improvement in performance.

### SUMMARY OF INVENTION

Thus, viewed from one aspect the invention provides a pharmaceutical composition comprising:

(A) at least one compound of formula (I):

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group;

or a pharmaceutically acceptable salt, or a hydrate or solvate thereof; and

(B) one or more calcineurin inhibitor partners such as pimecrolimus, tacrolimus or ciclosporin or a pharmaceutically acceptable salt, or a hydrate or solvate thereof, especially pimecrolimus or a pharmaceutically acceptable salt, or a hydrate or solvate thereof.

In a preferred embodiment, pimecrolimus or a pharmaceutically acceptable salt, or a hydrate or solvate thereof is the calcineurin inhibitor partner.

Viewed from another aspect the invention provides a pharmaceutical kit composition for simultaneous, in parallel, sequential or separate use comprising a first composition comprising at least one compound (I) as herein defined and a pharmaceutically-acceptable diluent or carrier, and a second composition comprising at least one compound (B) as the calcineurin inhibitor as herein defined such as pimecrolimus or a pharmaceutically acceptable salt, or a hydrate or solvate thereof and a pharmaceutically-acceptable diluent or carrier.

In particular, the invention relates to a pharmaceutical composition or kit as herein before defined in which the compound of formula (I) is:

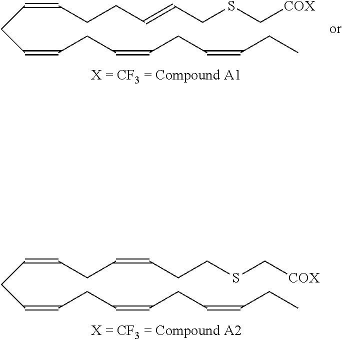

or a pharmaceutically acceptable salt, or a hydrate or solvate thereof. In particular, the calcineurin inhibitor partner (B) is pimecrolimus or a salt, hydrate or solvate thereof.

At least one other calcineurin inhibitor partner may be combined with the pimecrolimus to achieve intended results, for example, 1 or 2 of such compounds. Alternatively, the pimecrolimus (including a pharmaceutically acceptable salt, or a hydrate or solvate thereof) may be substituted by at least one other calcineurin inhibitor partner, for example, 1 or 2 of such other compounds (including salts, hydrates and solvates of such compounds).

Viewed from another aspect the invention provides a pharmaceutical composition as hereinbefore defined for use in the treatment or prevention of a skin disorder such as psoriasis or dermatitis.

Viewed from another aspect the invention provides a method of treating or preventing a skin disorder such as psoriasis or dermatitis in an animal subject, for example, a mammal such as rodent (mouse, rat, rabbit), monkey (or other non-human primate), pig or other laboratory animal used as a model to study skin disorders. Another suitable mammalian subject is a patient in need thereof. In one embodiment, the invention comprises administering to said subject (e.g. a human patient) an effective amount of a pharmaceutical composition as herein before defined.

Viewed from another aspect the invention provides a method of treating, such as reducing symptoms of, or preventing a skin disorder such as psoriasis or dermatitis, in a patient in need thereof comprising administering to said patient, preferably a human, an effective amount of at least one compound of formula (I) and simultaneously, in parallel, separately or sequentially administering to said patient an effective amount of at least one compound (B) (e.g., 1, 2 or 3 of such compounds) as herein defined. In sequential administration either compound can be administered first.

Viewed from another aspect the invention provides a method of treating, such as reducing symptoms of, or preventing a skin disorder such as psoriasis or dermatitis, in a patient in need thereof comprising:
(i) identifying a patient who has received either a compound of formula (I) or a compound (B);
(ii) administering to said patient an effective amount of either at least one of a compound (B) as herein defined or at least one compound of formula (I) as herein before defined so that said patient is administered with both at least one compound of formula (I) and at least one compound (B).

In preferred embodiments, 1, 2 or 3 of compound B will be suitable for use with the invention with 1 or 2 of compound B being preferred for many invention applications.

Viewed from another aspect the invention provides use of a pharmaceutical composition as hereinbefore defined in the manufacture of a medicament for treating or preventing a skin disorder such as psoriasis or dermatitis.

Viewed from another aspect the invention provides a process for the preparation of a pharmaceutical composition as hereinbefore defined comprising blending at least one compound of formula (I) or a pharmaceutically acceptable salt, or a hydrate or solvate thereof and at least one compound (B) or a pharmaceutically acceptable salt, or a hydrate or solvate thereof in the presence of at least one pharmaceutical excipient.

### Definitions

The term lower alkyl is used herein to refer to C1-6 alkyl groups, preferably C1-4 alkyl groups, especially C1-3 alkyl groups. These alkyl groups can be linear or branched, preferably linear.

In one embodiment, the invention relates to a pharmaceutical composition in which at least one compound (I) and at least one calcineurin inhibitor partner (e.g., 1, 2, or 3 of such compounds) are blended together in a single composition. The invention also relates to a pharmaceutical composition in the form of a kit in which the active compounds are provided in separate compositions but are designed for administration simultaneously, in parallel, separately or sequentially. Any method for treating or preventing a skin disorder as defined herein encompasses simultaneous, in parallel, separate or sequential administration of the active components or administration of the composition of the invention.

The pharmaceutical composition of the invention is a “combination”, which means either a fixed combination in one dosage unit form, or non fixed combination such as a kit of parts for combined administration where at least one compound of the formula (I) and at least one calcineurin inhibitor partner(s) (e.g., 1, 2 or 3 of such compounds) may be administered independently at the same time (e.g. in parallel) or separately within time intervals, especially where these time intervals allow that the combination partners show a cooperative and preferably a synergistic effect.

Thus a “pharmaceutical composition” as used herein means a product suitable for pharmaceutical use that results from the mixing, admixing or combining more than one active ingredient and includes both fixed and non-fixed combinations of the active ingredients. The term “fixed combination” or “fixed dose” means that the active ingredients, e.g. a compound of formula (I) and a calcineurin inhibitor partner such as pimecrolimus, are both administered to a patient simultaneously in the form of a single entity or dosage. The pharmaceutical composition can also be a “non-fixed combination” which means that the active ingredients, e.g. a compound of formula (I) and the combination partner are both administered to a patient as separate entities either simultaneously, in parallel, concurrently or sequentially with no specific time limits, wherein such administration provides therapeutically effective levels of the two compounds in the body of the animal in need thereof.

A calcineurin inhibitor partner as used herein means a synthetic or semisynthetic calcineurin inhibitor generally suitable for intended goals of the invention. Preferred calcineurin inhibitor partners include the following: pimecrolimus, tacrolimus or ciclosporin as well as pharmaceutically acceptable salts, hydrates or solvates thereof.

All discussion below relating to preferred compounds of the invention is equally applicable to both these aspects of the invention.

### DETAILED DESCRIPTION

This invention concerns a combination therapy of a compound of formula (I) and a calcineurin inhibitor, in particular pimecrolimus or a salt, hydrate or solvate thereof We have surprisingly found that this combination therapy results in synergy. Our results demonstrate a reduction in the proliferation and viability of HaCaT cells, the composition offering a larger decrease than could have been expected from the use of compounds individually, i.e. the combination of the compounds produces an overall effect that is greater than the sum of the individual elements.

### Pharmaceutical Composition of the Invention

The invention relies on the therapeutic combination of at least one compound of formula (I) or a pharmaceutically acceptable salt, or a hydrate or solvate thereof and at least one calcineurin inhibitor such as pimecrolimus or a pharmaceutically acceptable salt, or a hydrate or solvate thereof. The compound of formula (I) is

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a pharmaceutically acceptable salt, or a hydrate or solvate thereof.

The group R preferably comprises 5 to 9 double bonds, preferably 5 or 8 double bonds, e.g. 5 to 7 double bonds such as 5 or 6 double bonds. These bonds should be non-conjugated. It is also preferred if the double bonds do not conjugate with the carbonyl functionality.

The double bonds present in the group R may be in the cis or trans configuration however, it is preferred if the majority of the double bonds present (i.e. at least 50%) are in the cis configuration. In further advantageous embodiments all the double bonds in the group R are in the cis configuration or all double bonds are in the cis configuration except the double bond nearest the carbonyl group which may be in the trans configuration.

The group R may have between 10 and 24 carbon atoms, preferably 12 to 20 carbon atoms, especially 17 to 19 carbon atoms.

Whilst the R group can be interrupted by at least one heteroatom or group of heteroatoms, this is not preferred and the R group backbone preferably contains only carbon atoms.

The R group may carry up to three substituents, e.g. selected from halo, C₁₋₆ alkyl e.g. methyl, or C₁₋₆ alkoxy. If present, the substituents are preferably non-polar, and small, e.g. a methyl group. It is preferred however, if the R group remains unsubstituted.

The R group is preferably an alkylene group.

The R group is preferably linear. It preferably derives from a natural source such as a long chain fatty acid or ester. In particular, the R group may derive from AA, EPA or DHA.

Thus, viewed from another aspect the invention employs a compound of formula

R-L-CO—X (I′)

wherein R is a C₁₀₋₂₄ unsubstituted unsaturated alkylene group said group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group or a salt thereof

Ideally R is linear. R is therefore preferably an unsaturated C₁₀₋₂₄ polyalkylene chain.

The linking group L provides a bridging group of 1 to 5 backbone atoms, preferably 2 to 4 backbone atoms between the R group and the carbonyl, such as 2 atoms. The atoms in the backbone of the linker may be carbon and/or be heteroatoms such as N, O, S, SO, SO₂. The atoms should not form part of a ring and the backbone atoms of the linking group can be substituted with side chains, e.g. with groups such as C₁₋₆ alkyl, oxo, alkoxy, or halo.

Preferred components of the linking group are —CH₂—, —CH(C₁₋₆alkyl)-, —N(C₁₋₆alkyl)-, —NH—, —S—, —O—, —CH═CH—, —CO—, —SO—, —SO₂— which can be combined with each other in any (chemically meaningful) order to form the linking group. Thus, by using two methylene groups and an —S— group the linker —SCH₂CH₂— is formed. It will be appreciated that at least one component of the linker provides a heteroatom in the backbone.

The linking group L contains at least one heteroatom in the backbone. It is also preferred if the first backbone atom of the linking group attached to the R group is a heteroatom or group of heteroatoms.

It is highly preferred if the linking group L contains at least one —CH₂— link in the backbone. Ideally the atoms of the linking group adjacent the carbonyl are —CH₂—.

It is preferred that the group R or the group L (depending on the size of the L group) provides a heteroatom or group of heteroatoms positioned α, β, γ, or δ to the carbonyl, preferably β or γ to the carbonyl. Preferably the heteroatom is O, N or S or a sulphur derivative such as SO.

Highly preferred linking groups L therefore are —NH₂CH₂, —NH(Me)CH₂—, —SOCH₂—, or —COCH₂—

The linking group should not comprise a ring.

Highly preferred linking groups L are SCH₂, NHCl₂, and N(Me)CH₂.

Viewed from another aspect the invention employs a compound of formula (II)

R-L-CO—X (II)

wherein R is a linear C₁₀₋₂₄ unsubstituted unsaturated alkylene group said group comprising at least 4 non-conjugated double bonds;

L is —SCH₂—, —SOCH₂, or —SO₂CH₂—; and

X is an electron withdrawing group or a salt thereof.

The group X is an electron withdrawing group. Suitable groups in this regard include O—C₁₋₆ alkyl, CN, OCO₂-C₁₋₆ alkyl, phenyl, CHal₃, CHal₂H, CHalH₂ wherein Hal represents a halogen, e. g. fluorine, chlorine, bromine or iodine, preferably fluorine.

In a preferred embodiment the electron withdrawing group is CHal₃, especially CF₃.

Thus, preferred compounds of formula (I) are those of formula (III)

R-Y1-Y2—CO—X (III)

wherein R and X are as hereinbefore defined;

Y1 is selected from O, S, NH, N(C₁₋₆-alkyl), SO or SO₂ and

Y2 is (CH₂)ₙ or CH(C₁₋₆ alkyl); or

where n is 1 to 3, preferably 1.

More, preferred compounds of formula (I) are those of formula (IV)

R-Y1—CH₂—CO—X (IV)

wherein R is a linear C₁₀₋₂₄ unsubstituted unsaturated alkylene group said group comprising at least 4 non-conjugated double bonds;

X is as hereinbefore defined (e.g. CF₃); and

Y1 is selected from O, S, SO or SO₂.

Highly preferred compounds for use in the invention are depicted below.

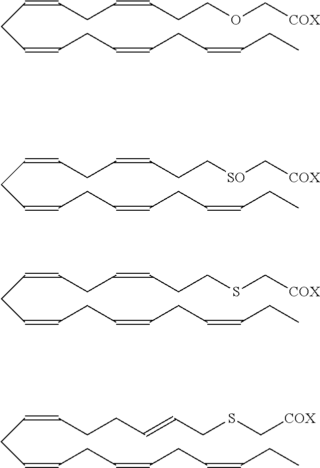

where X is as hereinbefore defined such as CF₃.

The following compounds are highly preferred for use in the invention:

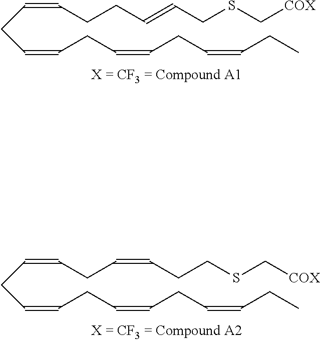

Salts, hydrates or solvates of any of these compounds could also be used. It will be appreciated that the pharmaceutical composition of the invention may comprise one or more than one compound of formula (I) as herein before defined, for example, 1, 2, or 3 of such compounds with 1 or 2 of such compounds being preferred for many invention applications.

### Compound (B)

The second component (compound B, i.e. the calcineurin inhibitor partner) of the composition of the invention is a calcineurin inhibitor such as pimecrolimus or a pharmaceutically acceptable salt, or a hydrate or solvate thereof. Pimecrolimus is a compound of formula:

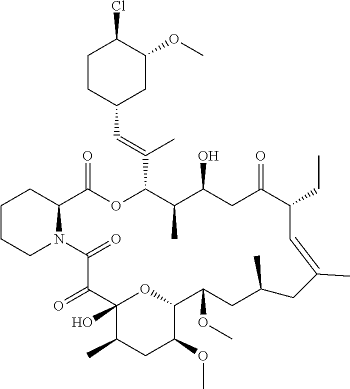 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](Cl)[C@H](OC)C3)[C@H](C)[C@@H](O)CC1=O)[C@H](C)C[C@@H]2OC]

In any composition of the invention the calcineurin inhibitor may be present in a salt or non salt form. In particular, in any composition of the invention, the compound (B) such as pimecrolimus may be present in a salt or non-salt form. If a salt form is used, any conventional salt form is possible. The salt may be a monosalt form, or disalt form, given the presence of multiple hydroxy groups on which salts can be formed.

Pimecrolimus is a known commercial product and any known commercial form of pimecrolimus can be used.

The use of pimecrolimus or a salt, hydrate or solvate thereof is especially preferred.

Whilst the invention is primarily described with reference to pimecrolimus, it is envisaged that other calcineurin inhibitors could also be combined with the compounds of formula (I) to form synergistic combinations, such as tacrolimus.

In one embodiment, the invention provides a composition comprising:

(A) a compound of formula (I):

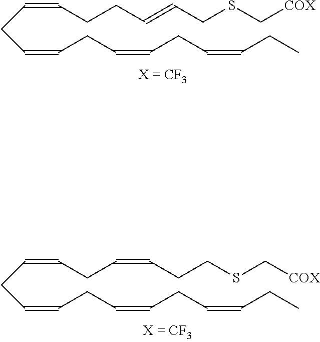

or a salt thereof; and

(B) a calcineurin inhibitor partner selected from the group consisting of pimecrolimus, tacrolimus or ciclosporin or a pharmaceutically acceptable salt, or a hydrate or solvate thereof, especially pimecrolimus or tacrolimus or a pharmaceutically acceptable salt, or a hydrate or solvate thereof, most especially pimecrolimus or a pharmaceutically acceptable salt, or a hydrate or solvate thereof.

Alternatively, and as discussed above, the composition of the invention could comprise pimecrolimus and comprise further calcineurin inhibitors to augment the properties of the composition of the invention. Suitable additional calcineurin inhibitors include tacrolimus or ciclosporin. The use of tacrolimus and pimecrolimus is an option therefore.

It is also within the scope of the invention to combine the composition of the invention with other compounds conventionally used in conjunction with calcineurin inhibitors. Such other agents include corticosteroids such as betamethasone or a pharmaceutically acceptable salt, or a hydrate or solvate thereof.

The amounts of each compound present in the composition of the invention are determined in molar terms, and the ratio of each is preferably calcineurin inhibitor to compound of formula (I) of 15:1 to 1:1 moles, such as 10:1 to 2:1 moles, or such as 7:1 to 3:1 moles, such as about 5:1 moles. The calcineurin inhibitor is therefore typically used in excess.

The amount of the compounds of the invention in the composition will often be determined by the physician depending on the dosage required.

### Skin Disorders

As noted above, the invention targets skin disorders, especially psoriasis and dermatitis. In particular, it is envisaged that the compositions of the invention may reduce inflammation and/or itchiness associated with the skin condition in question.

The combination therapy of the invention may have utility in treating a variety of different forms of dermatitis, such as atopic dermatitis or contact dermatitis. Thus, the compounds of the invention may be used to treat contact dermatitis such as allergic contact dermatitis or irritant contact dermatitis.

The nature of the allergan or irritant which causes the contact dermatitis can vary a lot and many people have different reactions to different allergans/irritants.

One of the most common causes of allergic contact dermatitis are plants of the Toxicodendron genus: poison ivy, poison oak, and poison sumac. Certain alkyl resorcinols such as bilobol found in Gingko biloba fruits are strong skin irritants. Other allergens include nickel, gold, balsam of Peru (Myroxylon pereirae), and chromium.

Common causes of irritant contact dermatitis are harsh (highly alkaline) soaps, detergents, and cleaning products. Irritant contact dermatitis can be divided into forms caused by chemical irritants and those caused by physical irritants. Common chemical irritants implicated include solvents (alcohol, xylene, turpentine, esters, acetone, ketones, and others); metalworking fluids (neat oils, water-based metalworking fluids with surfactants); latex; kerosene; ethylene oxide; surfactants in topical medications and cosmetics (sodium lauryl sulfate); alkalies (drain cleaners, strong soap with lye residues). Physical irritant contact dermatitis may most commonly be caused by low humidity from air conditioning. Also, many plants directly irritate the skin.

A further form of contact dermatitis is photocontact dermatitis. The skin condition is caused by exposure to ultraviolet light (320-400 nm UVA).

The invention may also lead to a treatment of atopic dermatitis. Atopic dermatitis is a type of eczema and is an inflammatory, chronically relapsing, non-contagious and itchy skin disorder.

Other less common forms of dermatitis to be treated include dermatitis herpetiformis, seborrheic dermatitis and stasis dermatitis. The composition may also be used to treat eczema. Other skin conditions to be treated include uticaria, and vitiligo.

Whilst the invention is primarily described with reference to skin disorders, the composition of the invention may also be used to treat rheumatoid arthritis. The use of the composition of the invention in the treatment or prevention of these conditions is also envisaged.

By treating or treatment is meant at least one of:

(i). inhibiting the disease i.e. arresting, reducing or delaying the development of the disease or a relapse thereof or at least one clinical or subclinical symptom thereof, or

(ii). relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the pharmaceutical compositions of the invention are used therapeutically, i.e. to treat a condition which has manifested rather than prophylactically. It may be that the pharmaceutical composition of the invention is more effective when used therapeutically than prophylactically.

The pharmaceutical composition of the invention can be used on any animal subject, in particular a mammal and more particularly a human or an animal serving as a model for a disease (e.g., rat, mouse, pig, monkey, etc.). For example, in one use a pharmaceutical composition of the invention is used as a positive control in the animal subject to test other compounds for activity and/or side effects.

In order to treat a disease an effective amount of the active pharmaceutical composition needs to be administered to a patient. A “therapeutically effective amount” means the amount of a pharmaceutical composition that, when administered to an animal for treating a state, disorder or condition, is sufficient to effect such treatment. The “therapeutically effective amount” will vary depending on the pharmaceutical composition, the disease and its severity and the age, weight, physical condition and responsiveness of the subject to be treated and will be ultimately at the discretion of the attendant doctor.

It may be that to treat skin disorders according to the invention that the pharmaceutical composition of the invention has to be readministered at certain intervals. Suitable dosage regimes can be prescribed by a physician.

The pharmaceutical composition of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The pharmaceutical composition can also contain other active components, e.g. other drugs for the treatment of skin disorders.

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The pharmaceutical compositions of the invention could also be formulated as nanoparticle formulations.

However, for the treatment of skin disorders, the pharmaceutical composition of the invention will preferably be administered topically. The pharmaceutical composition may therefore be provided in the form of a cream, gel, foam, salve or ointment.

The pharmaceutical composition of the invention may contain from 0.01 to 99% weight—per volume of the active material. The therapeutic doses will generally be between about 10 and 2000 mg/day and preferably between about 30 and 1500 mg/day of active components combined. Other ranges may be used, including, for example, 50-500 mg/day, 50-300 mg/day, 100-200 mg/day or active components combined.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The invention is described further below with reference to the following non-limiting examples and figures.

### DESCRIPTION OF FIGURES

FIG. 1: Co-treatment with cPLA2α inhibitor CompoundA1 and Pimecrolimus shows synergistic effects on decreasing keratinocyte cell proliferation and viability compared to each inhibitor alone. Average and standard deviation of 2-4 independent experiments performed in series of 8 technical replicates per treatment.

FIG. 2a/b: Dose response of Compound A1, pimcrolimus on immortalized keratinocyte cell line HaCat cell viability. Data presented are average and standard deviation of 1 independent experiments performed in series of 8 technical replicates per treatment.

FIG. 3: Co-treatment with Compound A1 and pimecrolimus shows synergistic effects on human keratinocyte cell viability compared to each inhibitor alone. Data presented are average and standard deviation of 1 independent experiments performed in series of 8 technical replicates per treatment.

### EXAMPLE 1

The following compounds were used in the Experiments:

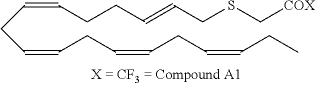

### Co-Treatment Compound A1 & Pimecrolimus

### Methods

### Cell Culture

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay

Cells were seeded in 96 well plates in fully supplemented medium at a density of 2500 cells per well. Following 72 hours of cultivation, the cells were starved of serum in 0.25% FBS/DMEM overnight to halt proliferation, synchronize and to increase cell sensitivity to treatment. On day 4, the cells were treated with cPLA2α inhibitor Compound A1 and immunomodulator Pimecrolimus (Karebay Biochem # Ki0907) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results

Co-treatment with cPLA2α inhibitor Compound A1 and immunomodulator Pimecrolimus shows synergistic effects on decreasing keratinocyte cell proliferation and viability compared to each inhibitor alone.

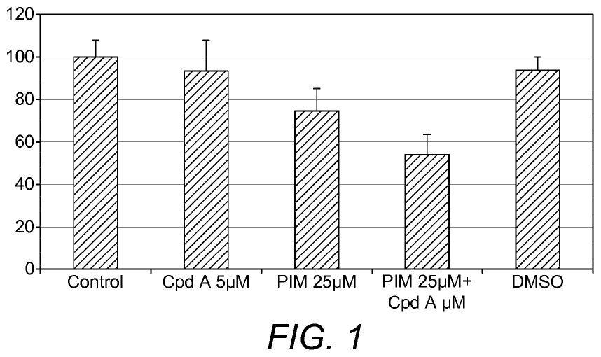

Initial experiments were performed to determine dose response of Compound A1 alone. The inhibitor slightly reduced cell proliferation and viability at 10 μM, whereas 5 μM did not show any affect (FIG. 1). On this basis, combination treatment experiments were designed in which sub-effective doses of the Compound A1 inhibitor and Pimecrolimus were combined.

Following 24 hours of treatment, 25 μM of Pimecrolimus resulted in a modest reduction of ˜15% and 5 μM Compound A1 alone showed little or no effect on reducing proliferation and viability of HaCaT cells. However, when combining sub-effective doses of Compound A1 and Pimecrolimus, a significant ˜45% reduction of proliferation and viability was observed (FIG. 1. This observed trend of synergistic effects on cell proliferation and viability indicates beneficial effects of co-treatment of on skin disorders.

Several key pathways are dysregulated in skin disorders such as psoriasis and atopic dermatitis. With this preliminary result, cPLA2α inhibitors represent a promising adjuvant treatment to other drugs in treatment of the inflammation and itching caused by a number of skin conditions such as psoriasis and dermatitis.

### EXAMPLE 2

### Methods: Cell Culture

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay

Cells were seeded in 96 well plates in fully supplemented medium at a density of 3000 cells per well. Following 72 hour of cultivation, the cells were starved of serum in 0.25% FBS/DMEM overnight to halt proliferation, synchronize the cells and to increase cell sensitivity to treatment. Next day, the cells were treated with Compound A1 and calcineurin inhibitor Pimecrolimus for 48 hours. Resazurin was added according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment.

### Results

Compound A1, calcineurin inhibitor Pimecrolimus shows dose response on immortalized keratinocyte cell line HaCat cell viability.

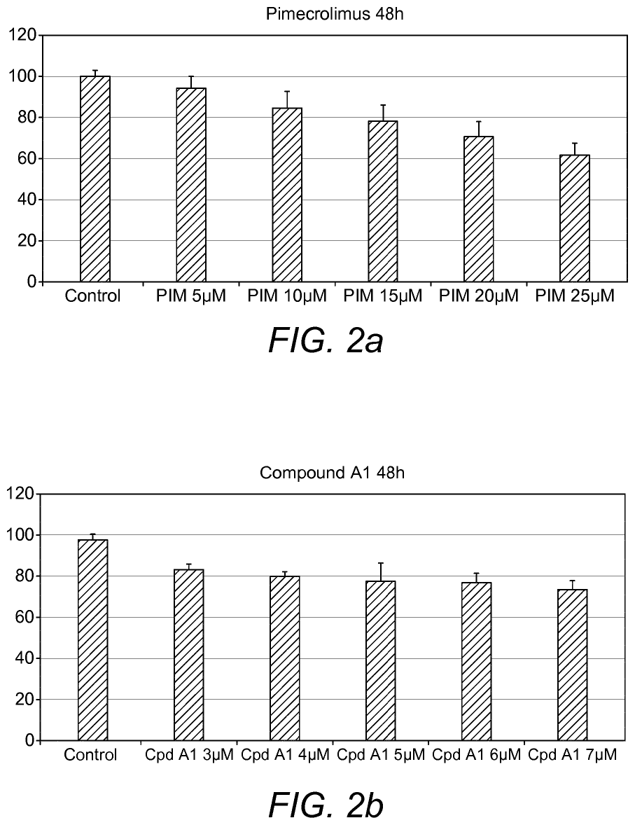

In this study experiments were performed to determine dose response over 48 hours and find out suboptimal doses of Compound A1 and Pimecrolimus resistant to Hacat. According to the results in the experiment, Compound A1 and pimecrolimus were found to be active inhibitors against viability of the cells, (FIG. 2a/b).

Co-treatment with Compound A1 and calcineurin inhibitor pimecrolimus shows synergistic effects on immortalized keratinocyte cell line HaCat cell viability compared to each inhibitor alone.

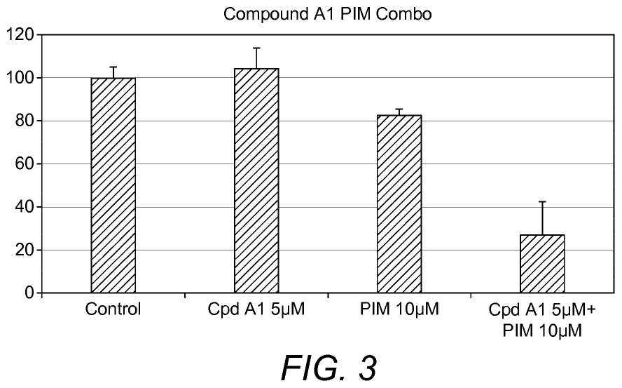

Initial experiments were performed to determine dose response of Compound A1 and pimecrolimus alone (FIG. 2a/b). On the basis of dose response, combination treatment was designed in which sub-optimal doses of Compound A1 (5 μM) and pimecrolimus (10 μM) were combined. Following 48 hours of treatment, 5 μM of Compound A1 and 10 μM of pimecrolimus shows 60% reduction of cell viability (FIG. 3). This observed trend of synergistic effects on cell viability indicate better beneficial effects of pimecrolimus combo with Compound A1 on skin disorders.

These results show that Compound A1 can be used as adjuvant treatment to other drugs in treatment of the inflammation and itching caused by a number of skin conditions such as psoriasis.

1. A pharmaceutical composition product comprising:
(A) at least one compound of formula (I):
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group;
or a pharmaceutically acceptable salt, or a hydrate or solvate thereof; and
(B) one or more calcineurin inhibitor partners such as pimecrolimus, tacrolimus or ciclosporin or a pharmaceutically acceptable salt, or a hydrate or solvate thereof, especially pimecrolimus or a pharmaceutically acceptable salt, or a hydrate or solvate thereof.

2. The pharmaceutical composition of claim 1 wherein the composition is a fixed combination or non-fixed combination.

3. A pharmaceutical composition as claimed in claim 1 for simultaneous, parallel, sequential or separate use comprising a kit comprising a first composition comprising at least one compound (I) and a pharmaceutically-acceptable diluent or carrier, and a second composition comprising at least one compound (B) and a pharmaceutically-acceptable diluent or carrier.

4. A composition as claimed in claim 1 wherein the compound (B) is pimecrolimus or tacrolimus, preferably pimecrolimus or a pharmaceutically acceptable salt, or a hydrate or solvate thereof.

5. A composition as claimed in claim 1 wherein the compound (B) is pimecrolimus or a pharmaceutically acceptable salt, or a hydrate or a solvate thereof.

6. A composition as claimed in claim 1 wherein in formula (I), the group X is CHal₃, preferably CF₃.

7. A composition as claimed in claim 1 wherein in formula (I), the group R is a linear unsubstituted C₁₀₋₂₄ unsaturated alkylene group comprising at least 4 non-conjugated double bonds.

8. A composition as claimed in claim 1 wherein L is —SCH₂—.

9. A composition as claimed in claim 1 wherein said compound of formula (I) has the formula: 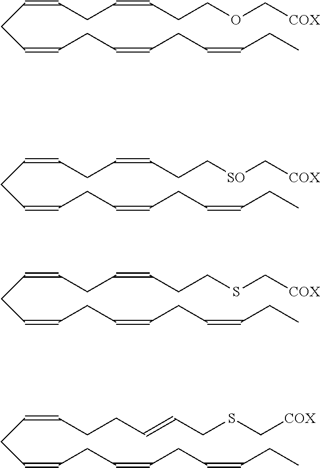
wherein X is as defined in claim 1, e.g. CF₃.

10. A composition as claimed in claim 1 wherein the compound of formula (I) is Compound A1 or Compound A2: 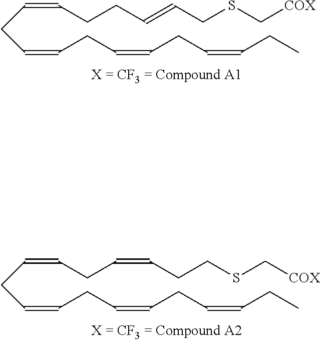
especially when compound (B) is pimecrolimus or a salt, hydrate or solvate thereof.

11. A composition as claimed in claim 1 wherein the molar ratio of compound (A) to (B) in the product is 15:1 to 1:1, preferably 10:1 to 2:1.

12. (canceled)

13. A method of treating, such as reducing symptoms of, or preventing a skin disorder such as psoriasis or dermatitis in a patient in need thereof comprising administering to said patient, preferably a human, an effective amount of a composition as claimed in claim 1.

14. A method of treating, such as reducing symptoms of, or preventing a skin disorder such as psoriasis or dermatitis in a patient in need thereof comprising administering to said patient, preferably a human, an effective amount of at least one compound of formula (I) and simultaneously, in parallel, separately or sequentially administering to said patient at least one compound (B), wherein formula (I) and compound (B) are as defined in claim 1.

15. A method of treating such as, reducing symptoms of, or preventing a skin disorder such as psoriasis or dermatitis, in a patient in need thereof comprising:
(i) identifying a patient who has received either a compound of formula (I) or a compound (B) respectively; and
(ii) administering to said patient an effective amount of either at least one compound
(B) or at least one compound of formula (I) so that said patient is administered with both a compound of formula (I) and a compound (B), wherein formula (I) and compound (B) are as defined in claim 1.

16. A method of treating, such as reducing symptoms of, or preventing a skin disorder such as psoriasis or dermatitis in an animal subject in need thereof comprising administering to said animal an effective amount of a composition as claimed in claim 1.

17. A method of treating, such as reducing symptoms of, or preventing a skin disorder such as psoriasis or dermatitis in an animal subject in need thereof comprising administering to said animal an effective amount of at least one compound of formula (I) and simultaneously, in parallel, separately or sequentially administering to said animal at least one compound (B), wherein formula (I) and compound (B) are defined in claim 1.

18. The method of claim 16, wherein the animal subject is a rodent, monkey, or a pig.

19. The method of claim 17, wherein the pharmaceutical composition or the effective amount of compound of formula (I) and compound (B) is used as a positive control.

20. (canceled)

21. The pharmaceutical composition of claim 1 comprising pimecrolimus or a salt, hydrate or solvate thereof optionally in combination with one or more additional calcineurin inhibitors such as tacrolimus or ciclosporin.

22. A pharmaceutical composition as claimed in claim 1 in a form suitable for topical administration, e.g. a cream, foam, gel or ointment.
